(19) United States
(12) Patent Application Publication
(10) Pub. No.: US 2025/0204825 A1
(43) Pub. Date: Jun. 26, 2025

(54) Reinforcement Learning-Driven Wearable Device and Uses Thereof

(71) Applicants: Amir Tofighi Zavareh, Hockley, TX (US); Yuexin Liu, College Station, TX (US); Ben Zoghi, Bryan, TX (US); Nicu Ahmadi, San Francisco, CA (US)

(72) Inventors: Amir Tofighi Zavareh, Hockley, TX (US); Yuexin Liu, College Station, TX (US); Ben Zoghi, Bryan, TX (US); Nicu Ahmadi, San Francisco, CA (US)

(73) Assignee: The Texas A&M University System, College Station, TX (US)

(21) Appl. No.: 18/990,141

(22) Filed: Dec. 20, 2024

Related U.S. Application Data
(60) Provisional application No. 63/613,985, filed on Dec. 22, 2023.

(51) Int. Cl. A61B 5/16; A61B 5/00; A61M 21/02; G06N 3/092

(52) U.S. Cl. CPC A61B 5/165; A61B 5/6802; A61B 5/7264; A61B 5/7267; A61M 21/02; G06N 3/092

(57) ABSTRACT

Provided herein are wearable devices and systems therewith configured with wearable sensors, a machine learning framework with a machine learning model, and reinforcement learning models. Also provided are methods utilizing the wearable devices and technologies for understanding and optimizing emotional states in a subject and for predicting anxiety in a subject.

### CROSS-REFERENCE TO RELATED APPLICATIONS

This non-provisional application claims benefit of priority under 35 U.S.C. § 119(e) of provisional application U.S. Ser. No. 63/613,985, filed Dec. 22, 2023, the entirety of which is hereby incorporated by reference.

### BACKGROUND OF THE INVENTION

### Field of the Invention

The invention relates to the fields of wearable devices and deep reinforcement learning. More specifically, the present invention relates to wearable devices to optimize emotional well-being via continuous monitoring of physiological indicators.

### Description of the Related Art

In the pursuit of holistic well-being, understanding the interplay between emotional states and physiological indicators is crucial (1). Emotions and vital signs can be used synergistically to enhance emotional balance and well-being (2). The intricate link between human emotions and the autonomic nervous system (ANS) has gained prominence, particularly in emotion research (3). The ANS plays a pivotal role in emotional response, a central tenet in contemporary emotion theories (4). Wearable devices like EMPATICA and FITBIT provide accurate measurements of parameters such as electrodermal activity, SpO₂, skin temperature, and heart rate (5, 6). These technologies validate physiological measurements and enhance understanding of the connections between emotional states, cognitive processes, and physiological responses (7-12).

Recent studies have highlighted the role of emotional intelligence in enhancing well-being and managing stress. Kaplan et al. (14) examined self-guided workplace interventions, “gratitude” and “social connectedness,” among 67 university employees. Results revealed gratitude improved positive affective well-being and reduced absenteeism. Di Fabio and Kenny (15) explored emotional intelligence's potential impact on well-being and performance among students, showing that trait emotional intelligence significantly contributed to both hedonic and eudaimonic well-being. Iqbal et al. (16) demonstrated the effectiveness of wearable sensors in monitoring stress through a pilot study and the creation of a stress-predict dataset, highlighting the significant physiological changes in response to stressors. Balconi et al. (17) explored the effects of technology-mediated mindfulness practice on stress, showing improvements in both psychophysiological and self-report measures of stress. While these studies provide valuable insights into individual aspects of emotional intelligence, mindfulness interventions, and wearable technology, they do not comprehensively examine their combined impact on emotional well-being.

The integration of Artificial Emotional Intelligence (AEI) into human-computer interaction (HCI) systems marks a significant advancement in the development of empathetic and responsive AI technologies. Artificial emotional intelligence, a burgeoning field within artificial intelligence, aims to equip machines with the capability to recognize, understand, and respond to human emotions (18). This advancement has the potential to transform emotional understanding in various settings, particularly in professional environments where emotions such as anxiety, stress, and excitement play a critical role in decision-making, performance, and overall well-being.

Previous studies have demonstrated that emotions significantly influence cognitive processes and behaviors, making the integration of artificial emotional intelligence into AI systems a natural progression for the development of more advanced, human-centric technologies (19). Despite the promising potential of artificial emotional intelligence, integrating physiological signals and biomarkers into AI systems to accurately interpret human emotions presents considerable challenges. Physiological signals such as electrocardiograms (ECG), galvanic skin responses (GSR), and electroencephalograms (EEG) offer non-invasive and real-time methods for monitoring emotional states. However, these signals are complex and can be influenced by various external factors, making accurate emotion recognition a challenging task. Furthermore, the development of algorithms that can handle the high-dimensional data generated by multimodal physiological signals remains an ongoing challenge (20, 21)

Despite significant advancements in artificial emotional intelligence and the use of physiological signals for emotion recognition, gaps remain in the application of these technologies in professional and educational contexts particularly in the application of these technologies in professional and educational contexts such as in the identification and management of anxiety as a secondary emotion. For example, there is a limited focus on anxiety as a secondary emotion, an underutilization of multimodal approaches in professional and educational contexts and inadequate real-time detection and intervention systems.

Thus, there remain unmet needs in the art for personalized strategies for improving emotional well-being. Particularly, the art is deficient in wearable devices that incorporate advanced machine learning to optimize emotional well-being via continuous monitoring of physiological indicators and of suggestions of personalized mindfulness-based emotional intelligence practices.

### SUMMARY OF THE INVENTION

The present invention is directed to a method for understanding and optimizing emotional states in a subject using a wearable device. In this method, physiological data is collected from the subject using the wearable device and self-reported emotional states are received from the subject via a user interface in the wearable device. The physiological data is processed using a machine learning model to identify patterns or relationships or a combination thereof between the physiological data and the self-reported emotional states, where as a machine learning algorithm is developed, reliance on self-reported emotions decreases as processing the physiological data increases. A reinforcement learning model is employed where the states in reinforcement learning algorithms are the emotions calculated using a developed machine learning algorithm. The present invention is directed to a related method further comprising improving emotional intelligence in the subject by optimizing at least one intervention that is implemented within the reinforcement learning model.

The present invention is further directed to a system comprising at least one wearable device. The wearable device is configured to continuously measure physiological signals of a subject wearing the wearable device where the wearable device comprises a machine learning-based framework, to assess emotional states of the subject based on the physiological signals via the machine learning-based framework, to detect deviations in emotional states and to utilize the results of at least one reinforcement learning model to suggest, in real time, activities to alter the subject's emotion, to enhance an emotional quotient or to reduce stress over time or a combination thereof. The present invention is directed to a related system further configured to utilize the results of at least one reinforcement learning model to suggest, in real time, food intake to alter the subject's emotion, to induce happiness or to reduce stress over time or a combination thereof. The present invention is directed to another related system further configured to utilize the results of at least one reinforcement learning model to suggest, in real time, physical or athletic activities to alter the subject's physical outcome over time comprising muscle mass or body mass index (BMI) or a combination thereof.

The present invention is directed further to a method for predicting anxiety in a subject. In this method, physiological signals are continuously measured in the subject wearing a wearable device that comprises a plurality of sensors, a machine learning-based framework and a reinforcement learning model. Emotional states are assessed based on the measured physiological signals where a deviation in a stress-inducible emotion in the subject is indicative of an onset of anxiety. The present invention is directed to a related method further comprising suggesting a personalized emotional intelligence (EI) intervention based on results from the reinforcement learning model to reduce or prevent anxiety in the subject.

Other and further aspects, features, benefits, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others that will become clear, are attained and can be understood in detail, more particular descriptions of the invention briefly summarized above may be had by reference to certain embodiments thereof that are illustrated in the appended drawings. These drawings form a part of the specification. It is to be noted, however, that the appended drawings illustrate preferred embodiments of the invention and therefore are not to be considered limiting in their scope.

FIG. 1 is a schematic of the Reinforcement Learning Framework illustrating the components of the reinforcement learning model. These include: 1) The agent, representing human beings; 2) The environment, corresponding to participants' daily life, 3) State, denoting participants' emotional status; 4) Action, encompassing interventional practices aimed to improve one's emotional intelligence, that is, mindfulness practices, breathing practices, mental imagery, and body scanning, and 5) Reward, signifying the score change associated with each action towards the predefined goal.

FIG. 2 is a schematic of the architectural structure of Return Decomposition for Delayed Rewards (RUDDER) that addresses the challenge of delayed rewards in reinforcement learning in the context of mental well-being improvements. The framework employs return decomposition techniques to disentangle and assign credit to intermediate actions within the delay horizon, facilitating improved learning and decision-making processes.

FIG. 3 is a heatmap showing the correlations between the Perceived Stress Scale (PSS), WHO-5 Well-Being Index (WHO-5) and the biweekly means of various biomarkers.

FIGS. 4A-4B show the model performance comparisons for the PSS Label Prediction (FIG. 4A) and the WHO-5 Label Prediction (FIG. 4B). RF (Random Forest), GB (Gradient Boosting), AB (AdaBoost), ET (Extra Trees), ENS (Ensemble of Multiple Models), RF (SF) (Random Forest with Selected Features), ET (SF) (Extra Trees with Selected Features).

FIG. 5 illustrates the emotion wheel and emotion emojis used by participants when prompted for their daily reflections used to create the labels to train a machine learning algorithm that detects the emotional status from the physiological signal.

FIG. 6 illustrates the web application for real-time logging that is an alternate method for collecting emotions from participants and train a machine learning algorithm to detect emotional status from physiological signals.

FIG. 7 illustrates the preprocessing of physiological data from the EMPATICA Embrace Plus watch collected from participants over a week. The data is used to train the machine learning algorithm to detect emotions using physiological data.

FIG. 8 shows the anxiety distribution as frequencies of emotions.

FIG. 9 shows the permutation importance scores for each physiological signal that was used to detect anxiety episodes using a machine learning algorithm.

FIG. 10 is a model comparison showing Precision-Recall curves of machine learning models that use physiological signals as features and anxiety as the feature.

FIG. 11 is a model comparison showing learning curves of multiple machine learning models that use physiological signals as features and anxiety as the feature.

FIG. 12 shows the best model for the learning curve of the best machine learning model that usea physiological signals as features and anxiety as the feature

FIG. 13 shows the best model for the precision-recall curve random forest machine learning model with hypertuning and selected features that use physiological signals as features and anxiety as the feature.

### DETAILED DESCRIPTION OF THE INVENTION

As used herein, the articles “a” and “an” when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, components, method steps, and/or methods of the invention. It is contemplated that any composition, component or method described herein can be implemented with respect to any other composition, component or method described herein.

As used herein, the term “or” in the claims refers to “and/or” unless explicitly indicated to refer to alternatives only or the alternatives are mutually exclusive, although the disclosure supports a definition that refers to only alternatives and “and/or”.

As used herein “another” or “other” may mean at least a second or more of the same or different claim element or components thereof.

As used herein, the terms “comprise” and “comprising” are used in the inclusive, open sense, meaning that additional elements may be included.

As used herein, the terms “consist of” and “consisting of” are used in the exclusive, closed sense, meaning that additional elements may not be included.

As used herein, the terms “wearable device” and “wearable” are used interchangeably.

As used herein, the terms “participant” and “subject” are used interchangeably.

As used herein, the terms “emotional quotient” or “EQ” and “emotional intelligence” or “EI” are used interchangeably.

In one embodiment of the present invention, there is provided a method for understanding and optimizing emotional states in a subject using a wearable device, comprising a) collecting physiological data from the subject using the wearable device; b) receiving self-reported emotional states from the subject via a user interface in the wearable device; c) processing the physiological data using a machine learning model to identify patterns or relationships or a combination thereof between the physiological data and the self-reported emotional states; wherein as a machine learning algorithm is developed, reliance on self-reported emotions decreases as processing the physiological data increases; and d) employing a reinforcement learning model; wherein the states in reinforcement learning algorithms are the emotions calculated using the developed machine learning algorithm.

Further to this embodiment, the method comprises integrating a Return Decomposition for Delayed Rewards (RUDDER) framework into the reinforcement learning model to address issues of delayed rewards. In this further embodiment RUDDER may utilize return decomposition techniques to assign credit to intermediate actions within a delay horizon.

In another further embodiment, the method comprises improving emotional intelligence in the subject by optimizing at least one intervention that is implemented within the reinforcement learning model. In this further embodiment, the at least one intervention may be an activity comprising mindfulness, mental imagery, breathing, body scanning, exercise or dietary changes.

In all embodiments, the wearable device may comprise a watch, a ring, a patch or other wearable technology. Also, in both embodiments the machine learning model may comprise Convolutional Neural Networks (CNNs) configured for deciphering complex, time-dependent associations present in physiological signals; Long Short-Term Memory (LSTM) and Recurrent Neural Networks (RNNs) configured for sequence and temporal pattern recognition; and Random Forests and Support Vector Machines (SVMs) for identifying nonlinear relationships within multidimensional datasets. In addition, the wearable device may comprise at least a PPG sensor, an ECG sensor, a bioimpedance sensor, a gyroscope, an accelerometer, a temperature sensor, and a force sensor to measure physiological signals comprising pulse rate, pulse rate variability, SpO_{2,}electrodermal activity, skin temperature, or actigraphy data or a combination thereof.

In both embodiments in step d), the reinforcement learning model may comprise activities defined as EQ enhancing activities, consuming nootropics, adopting new dietary habits, physical exercises, or other activity effective to improve mental status, or a combination thereof. Particularly, in step d), the reinforcement learning model may comprise rewards defined as trends in mental well-being metrics that are perceived stress test, WHO-5, concentration trends of mental status-related biomarkers in the blood, or physical attributes of the subject or other metric for assessing trends of mental or physical status over time. In an aspect the reinforcement learning model scores the activities based on the rewards observed.

In another embodiment of the present invention, there is provided a system comprising at least one wearable device configured to continuously measure physiological signals of a subject wearing the wearable device, where the wearable device comprises a machine learning-based framework; assess emotional states of the subject based on the physiological signals via the machine learning-based framework; detect deviations in emotional states; and utilize the results of at least one reinforcement learning model to suggest, in real time, activities to alter the subject's emotion, to enhance an emotional quotient (EI) or to reduce stress and improve overall well-being over time or a combination thereof.

Further to this embodiment, the system is configured to utilize the results of at least one reinforcement learning model to suggest, in real time, food intake to alter the subject's emotion, to induce happiness or to reduce stress over time or a combination thereof. In another further embodiment, the system is configured to utilize the results of at least one reinforcement learning model to suggest, in real time, physical or athletic activities to alter the subject's physical outcome over time comprising muscle mass or body mass index (BMI) or a combination thereof.

In yet another embodiment of the present invention, there is provided a method for predicting anxiety in a subject, comprising measuring continuously physiological signals in the subject wearing a wearable device comprising a plurality of sensors, a machine learning-based framework and a reinforcement learning model; cleaning and concatenating physiological data derived from the physiological signals from the wearable device to form a complete physiological state; assessing emotional states based on the measured physiological signals by merging the physiological data with corresponding emotional annotations over a predefined window of time via a method of data synchronization; detecting a deviation in a stress-inducible emotional state in the subject indicative of an onset of anxiety; and performing a machine learning analysis by selecting features from annotated physiological data and training a model to evaluate the emotional state, thereby predicting the onset of anxiety therefrom.

Further to this embodiment the method comprises suggesting a personalized emotional intelligence intervention based on results from the reinforcement learning model to reduce or prevent anxiety in the subject. In this further embodiment the emotional intelligence intervention may be mindfulness.

In both embodiments the cleaning and concatenating step may comprise handling missing data, aggregating cleaned physiological data and merging the physiological data with a participant ID and timestamp. Also in both embodiments, the subject may suffer from post traumatic stress syndrome. In addition, the subject may be in the military, is a veteran, is a civilian or is in an educational setting or a professional setting or a combination thereof. An example of a stressful situation is post traumatic stress syndrome.

Provided herein is wearable sensor technology that utilizes wearable devices or wearables to integrate wearable sensors, advanced machine learning (supervised, unsupervised, and reinforcement) models, and mindfulness interventions to provide a comprehensive solution for enhancing emotional well-being, alleviating stress, and offering individualized support. The wearable device continuously monitors physiological data. Non-limiting examples of physiological data are vital indicators like heart rate, blood oxygen saturation (SpO2), sleep patterns, electrodermal activity (EDA), skin conductance level (SCL), and temperature. These wearable devices operate throughout both daytime and nighttime, collecting data at high-frequency intervals. Providing real-time, continuous monitoring of physiological responses provides emotion assessment, overcoming the subjectivity inherent in emotional expressions. This enables a more accurate measurement of emotional states in real-life contexts.

During use of the wearable device participants engage in a weekly self-reporting process or emotion labeling where they identify their emotional states using a predefined set of primary emotions. They tag specific events when their emotional states deviate from their baseline, providing context to the physiological data and bridging the gap between subjective experiences and objective physiological data within the context of real-life events. Examples of self-monitored and self-assessed basic emotions are happiness, sadness, fear, anger, disgust, and surprise each of which may be broken down into more specific levels of feelings and may be selected from an emotion wheel.

The machine learning models are configured with algorithms such as, but not limited to, Convolutional Neural Networks (CNNs), Long Short-Term Memory (LSTM), Recurrent Neural Networks (RNNs), Random Forests, and Support Vector Machines (SVMs) to interpret the physiological data. The algorithms are configured to decipher complex, time-dependent associations present in physiological signals to uncover patterns that might be elusive to human perception alone.

A reinforcement learning model is configured for personalized emotional balance recommendations based on the user's physiological data and self-reported emotions. The Q-learning model uses accumulated data and feedback to refine and personalize recommendations. Additionally, the RUDDER framework is integrated to address any challenges of delayed rewards in reinforcement learning. The model provides tailored suggestions and continuously refines recommendations based on feedback so that interventions are personalized and effective for each individual.

The wearable sensor technology is utilized to evaluate the impact of diverse mindfulness interventions, such as, but not limited to, mindful yoga, breath, physical exercise, coloring, mental imaging, body scanning, and reading, that are specifically designed to improve a subject's EQ competencies, and to reduce stress as the result and to assess participants' overall well-being and emotional equilibrium. By assessing the efficacy of various mindfulness practices, the wearable technology ensures that interventions are evidence-based and have a tangible positive impact on emotional well-being. The wearable sensor technology is useful in predicting the onset of stress, such as anxiety in the subject that may occur in various situations. The subject may be in the military, be a veteran, a civilian. The subject may be in an educational environment or in a professional environment.

Also provided are methods to predict an onset of anxiety in a subject using the wearable devices. The machine learning framework in the wearable device is configured to develop an algorithm that performs a machine learning analysis on a concatenation of cleaned physiological data from continuously received physiological signals and merges the same with corresponding emotional annotations of emotional states self-assessed by a subject via data synchronization. Deviations in stress-inducible emotional states may be detected and used to train a model to evaluate the emotional state and predict an onset of anxiety. Moreover, emotional intelligence interventions, such as, but not limited to, mindfulness, may be suggested as a therapy. A non-limiting example of a subject in need of such therapy is one suffering from post traumatic stress syndrome.

The following examples are given for the purpose of illustrating various embodiments of the invention and are not meant to limit the present invention in any fashion.

### Example 1

Correlation between emotions and vital signs: Methods

### Participant Recruitment

Participants are recruited according to the following criteria:
1) Inclusion criteria:
   A total of 50-65 participants.
   Both male and female individuals
   Age 18 years or older and enrolled in a graduate education program.
2) Exclusion criteria:
   Individuals unable to wear the EmbracePlus for a minimum of 8 hours per day.
   Individuals aged 17 years or younger.

### Emotion Detection

The correlation between emotions and vital signs is examined. Changes in vital signs, such as heart rate and blood pressure, are explored as potential indicators of various emotional states or states of mind.

1) Data Collection: participants are each equipped with a wearable device, such as an EmbracePlus (Empatica S.R.L.), to continuously monitor physiological data. This includes vital indicators such as heart rate, blood oxygen saturation (SpO2), sleep patterns, electrodermal activity (EDA), skin conductance level (SCL), and temperature, as shown in Table 1. The wearables operate throughout both the daytime and nighttime, collecting data at high-frequency intervals. Participants wear the devices for a minimum of 8 hours daily, ensuring a thorough and holistic capture of their physiological responses.

TABLE 1
Data sources

| Data | Feature Collected | Collecting Method |
|---|---|---|
| Embrace Plus | Temperature | Actigraphy - a non-invasive |
| | IBI (Systolic Peaks) | Technique that measures by |
| | Accelerometer | Means of a watch-like device |
| | Blood Volume Pulse | that can be worn for |
| | Blood oxygen saturation | prolonged periods of time. |
| | (SpO2) | |
| | Sleep detection | |
| | Electrodermal Activity (EDA) | |
| | Skin Conductance Level | |
| | (SCL) | |
| | Respiratory Rate | |
| | Pulse Rate | |
| | Pulse Rate Variability | |
| | Activity Classification | |
| | Activity Counts Steps | |
| WHO-5 | 5 question self-report measure | Web-based survey (i.e., |
| | of well being | Qualtrics or Google Forms) |
| PSS | 10 question self-report | Web-based survey (i.e., |
| | measure of stress | Qualtrics or Google Forms) |
| Open-ended Survey: | Free responses targeting | Web-based survey (i.e., |
| Journal prompts | thoughts, emotions, and | Qualtrics or Google Forms) |
| | physical changes related to a | |
| | specific (participant-identified) | |
| | event | |

2) Emotion Labeling: additionally, participants engage in a weekly self-reporting process where they are prompted to identify their emotional states using a predefined set of primary emotions, such as, happiness, surprise, anger, sadness, disgust, and fear. This weekly reflection allows participants to recap their experiences over the past week and discern their emotional fluctuations. Notably, participants tag specific events when their emotional states deviate from their baseline, and during these flagged moments, they accurately identify the primary emotion experienced. This approach offers a nuanced understanding of emotions within the context of real-life events and variations throughout the week.

3) Machine learning Model I Employment: Utilizing the physiological data as the independent variable and self-reported emotional states as dependent variable, the machine learning model is engineered to unveil intricate relationships and connections between vital signs and emotional states. Notably, features such as heart rate variability, blood pressure fluctuations, and electrodermal activity are meticulously engineered to encapsulate the essence of emotional responses. Through this process, the model seeks to unearth concealed patterns that might be elusive to human perception alone. The chosen algorithms for this endeavor encompass a fusion of approaches proven adept in similar domains.

Convolutional Neural Networks (CNNs) (22), Long Short-Term Memory (LSTM) (23), and Recurrent Neural Networks (RNNs) emerge as compelling contenders due to their prowess in sequence and temporal pattern recognition. These networks, adapted from their successful applications in image and sequential data analysis, hold promise in deciphering complex, time-dependent associations present in physiological signals.

Additionally, Random Forests (24) and Support Vector Machines (SVMs) (25) are harnessed for their proficiency in identifying nonlinear relationships within multidimensional datasets. As outlined by previous studies in emotion detection, these algorithms demonstrate their potential to dissect and interpret intricate physiological data, aligning with the objective of uncovering meaningful correlations between vital signs and emotional states.

### Mindfulness Intervention Effectiveness Evaluation

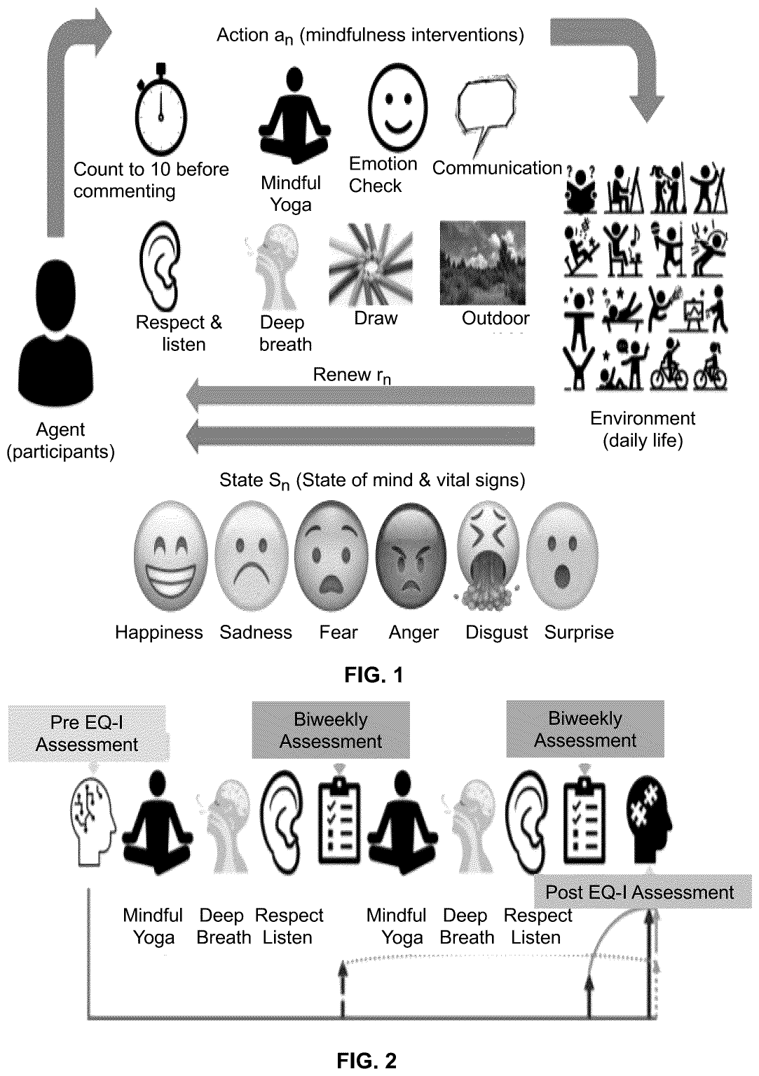

Diverse mindfulness intervention, such as mindful yoga, breathing, physical exercise, body scanning, imagery, coloring, and reading, as shown in FIG. 1, the impact on participants' overall well-being, emotional equilibrium, and the augmentation of their Emotional Intelligence are investigated. Notably, participants have previously completed the EQ-i 2.0 (26) assessment to establish their baseline EQ competency. Subsequently, participants collaborate with a designated EQ coach to target a specific EQ competency for enhancement. Through this collaboration, a curated list of 3 to 5 activities tailored to bolster the chosen EQ competency is devised. These activities encapsulate a spectrum of mindfulness techniques, such as meditation practices and focused breathing exercises, all geared towards fostering EQ development. The assessment of the effectiveness of these interventions is multifaceted, encompassing both subjective and objective measures. Participants' self-reported well-being and emotional balance are captured through standardized well-being indices, such as the WHO 5, and the Perceived Stress Scale (PSS) administered on a bi-weekly basis. In parallel, the coach-led interventions' impact on the chosen EQ competency is tracked through participants' weekly completion of open-ended questionnaires, providing insight into the efficacy of the mindfulness practices. This phase spans approximately 21 months.

1) Intentional Practice: participants are introduced to different mindfulness techniques, such as meditation, EQ-based strategy and breathing exercises, through guided sessions.

2) Pre-and Post-Intervention Assessment: participants complete well-being and emotional balance self-report measures (e.g., WHO 5 and Perceived Stress Scale as shown in Table. 1, and use the pre-post prompt to capture the emotion) before starting the intervention. Participants access the well-being and stress surveys and open-ended questionnaire via web-based platforms, at a self-selected time, on a bi-weekly or weekly basis respectively. NVivo is utilized for qualitative analysis.

3) Mindfulness Sessions: participants engage in regular mindfulness sessions over the course of several months. Subsequently, in collaboration with a designated coach, participants focus on enhancing a specific EQ competency. This collaborative effort results in a customized list of 3 to 5 activities designed to bolster the targeted EQ competency.

4) Machine learning Model II Implementation: the assessment of intervention effectiveness employs a comprehensive approach encompassing subjective and objective measurements. Standardized well-being indices, such as the WHO 5 and the Perceived Stress Scale (PSS), are administered bi-weekly to capture participants' self-reported well-being and emotional equilibrium. Concurrently, the impact of coach-led interventions on the chosen EQ competency is gauged through participants' weekly completion of open-ended questionnaires, which can be utilized for mindfulness practices' efficacy. To facilitate the transition from qualitative to quantitative data, NVivo (27) is employed. Feature extraction is subsequently followed by machine learning analysis to evaluate intervention effectiveness. Prominent algorithms, including Random Forest, Support Vector Machines, and Neural Networks, are among the potential choices to uncover patterns and correlations within the acquired data.

### Personalized Recommendation System Development

A reinforcement learning model, where the schematic details of its architecture and operation are depicted in FIG. 1, is designed to offer individualized recommendations aimed at enhancing participants' overall well-being and emotional equilibrium. The model dynamically adjusts its recommendations based on individual responses and feedback. This developmental process spans the entirety of the study.

1) Customized Recommendations: The reinforcement learning model analyzes participants' physiological data, self-reported emotions, and intervention responses to generate tailored suggestions.

2) Continuous Feedback Loop: Participants receive suggestions based on their data and subsequently provide feedback on the effectiveness of the suggestions.

3) Reinforcement Learning Model: Q-learning (28) model is cultivated using the accumulated data and feedback to iteratively refine and personalize recommendations for each participant, drawing upon the effectiveness of Q-learning to optimize the decision-making process, as depicted in FIG. 1.

4) RUDDER Integration: The reinforcement learning model incorporates the RUDDER: return decomposition for delayed rewards framework, utilizing delayed rewards and improved training techniques to enhance the efficiency and efficacy of the recommendation system (FIG. 2).

### Example 2

### Identification of Specific Physiological Patterns Linked to Anxiety

A cohort of professional students, each with a minimum of four years of technical work experience and a bachelor's degree, primarily in STEM fields participated in the study. To collect data, participants were provided with the EMPATICA EmbracePlus smartwatch, a device well-suited for continuous monitoring of key physiological biomarkers including heart rate, electrodermal activity (EDA), respiratory rate, peripheral skin temperature, step counts, activity counts and respiration rate. The device's advanced capabilities allow for precise tracking of the autonomic nervous system's response to emotional stimuli throughout the study. Baseline data was collected at the start of a residency week through an in-person session with all participants. Additionally, participants completed the Perceived Stress Scale (PSS) and the World Health Organization Well-Being Index (WHO-5) during the residency week to establish a baseline of perceived stress levels and well-being. After the residency week, participants transitioned to providing weekly emotional reflections, by an in-house designed app, supplemented by bi-weekly Perceived Stress Scale (PSS) and World Health Organization Well-Being Index (WHO-5) assessments to monitor ongoing well-being. The web application was available throughout the semester for continuous real-time emotional logging, enabling the study to capture a comprehensive dataset of emotional states over time.

### Correlation Analysis

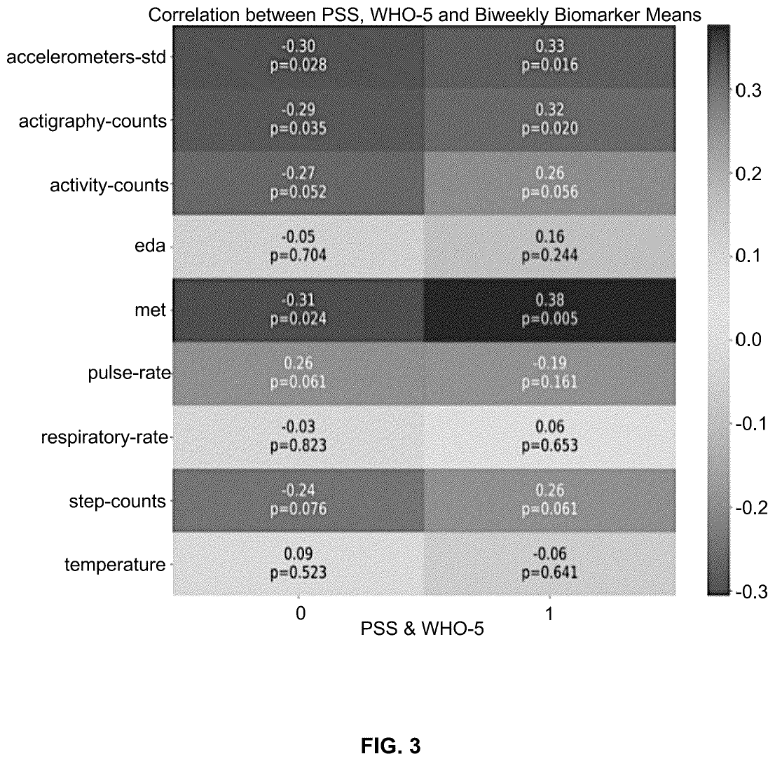

The EMPATICA EmbracePlus signals were measured continuously over the course of one semester. Bi-weekly PSS and WHO-5 scores were categorized into specific groups to assess stress and well-being levels. FIG. 3 demonstrates the correlation between average physiological signals every two weeks and the PSS and WHO-5 results. A significant negative correlation was observed between physical activity-related signals, such as accelerometer standard deviation, actigraphy count, activity count, metabolic equivalent of task (MET), step counts, and stress levels. This finding aligns with expectations, suggesting that increased physical activity is associated with PNS activation and lower perceived stress levels. Additionally, the average bi-weekly heart rate (HR) showed a significant positive correlation with stress levels, as anticipated. However, no significant correlation was found between electrodermal activity (EDA) and stress or between skin temperature and stress, possibly due to the environmental influence on these signals. New techniques will be proposed to control the environmental effects.

### Categorization and Labeling

PSS and WHO-5 scores were categorized into groups to create labels for machine learning classification. The categories were as follows: 1) PSS: Low Stress: Scores between 0 and 13, Moderate Stress: Scores between 14 and 26, High Perceived Stress: Scores between 27 and 40, Unknown: Missing reports from participants. 2) WHO-5: Indicative of Depression: Scores≤28 suggest possible depression, Poor Well-being, May Indicate Depression: Scores between 29 and 50, Significant Change in Well-being: Indicated by a notable percentage change from the previous measurement (e.g., >10%), Good Well-being: Scores>50 suggest good mental well-being.

### Machine Learning Model Development

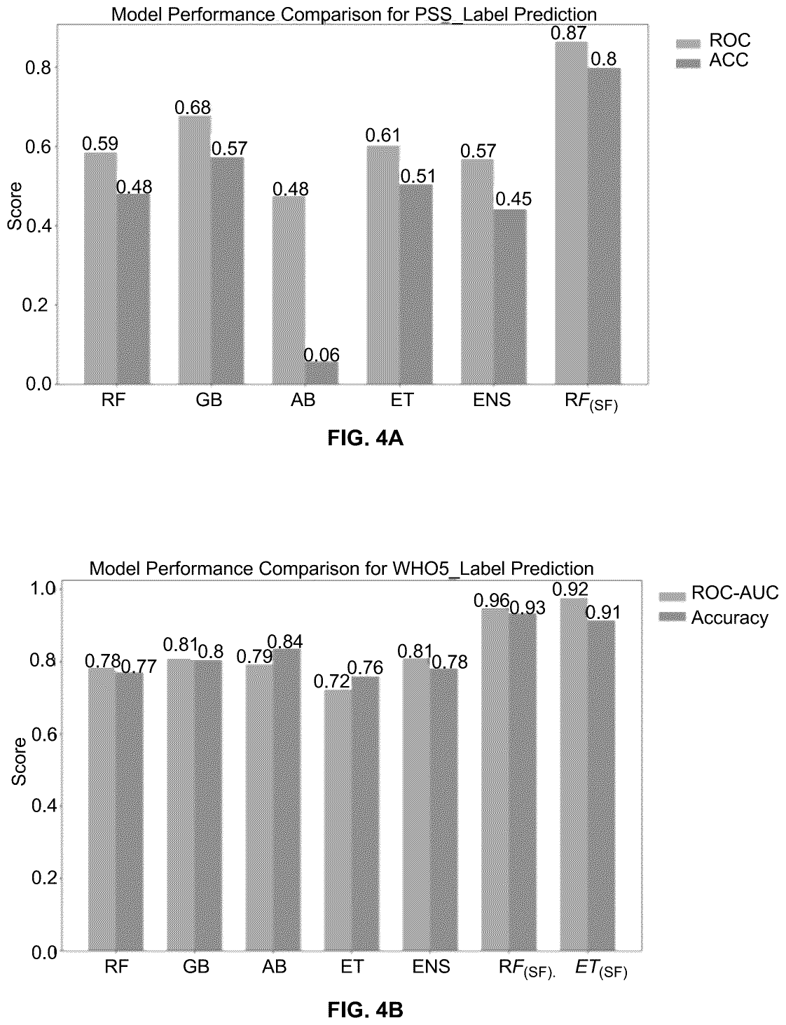

The categorized PSS and WHO-5 scores were used as labels, while the average weekly physiological signals served as features to train multiple machine learning algorithms. The participants' stress and well-being levels were classified based on their physiological data. Initial results showed promising classification accuracy, particularly for distinguishing between low, moderate, and high stress levels. The PSS, and WHO5 classes were detected with an accuracy and AUC of >80 for PSS and >90 for WHO5 indicating the ability of physiological signals, measured in real-life scenarios, to discern different levels of stress and overall well-being (FIGS. 4A-4B).

### EXAMPLE 3

### Machine Learning Model for Detection of and Prediction of Anxiety

The machine learning model focuses on monitoring physiological signals in a cohort of professional students, each with a minimum of four years of technical work experience and a Bachelor's degree, preferably in STEM fields. By analyzing real-time physiological data and correlating it with self-reported emotional states, stress levels, and overall well-being, the goal is to identify patterns linked to anxiety and improve emotion detection accuracy through machine learning techniques.

At the outset of the semester, an in-person session was conducted with all participants during which baseline physiological data was collected. This session was crucial for establishing a reference point for each participant's physiological signals, which would later be compared against data collected during and after the residency week.

The EmbracePlus was selected as it is one of the most advanced smartwatches for continuous health monitoring for its ability to capture a wide range of physiological data with high precision. Equipped with medical-grade sensors, the EmbracePlus offers comprehensive access to key physiological biomarkers, HRV, EDA, respiratory rate (RR), and peripheral skin temperature, essential for assessing autonomic nervous system responses to emotional stimuli. Table 2 describes each biomarker.

TABLE 2
Biomarkers from EMPATICA sensors

| Biomarker | Explanation | Common Value/Range |
|---|---|---|
| Accelerometer | Measures physical motion and | Varies widely based on |
| Readings | acceleration | activity level |
| Actigraphy Counts | Tracks movement, often used | Varies based on activity; |
| | to study sleep and activity | higher counts indicate more |
| | patterns | movement |
| EDA | Measures skin conductance, | Baseline varies; increases |
| | reflecting sympathetic nervous | with arousal |
| | system activity | |
| Metabolic Equivalent | A unit to estimate the amount | 1 MET at rest; higher during |
| (MET) | of energy used by the body | physical activity (e.g., 3-6 |
| | during physical activity, | METs for moderate activities) |
| | compared to resting state | |
| PR | Number of heartbeats per | Resting rate: 60-100 bpm |
| | minute | (adults) |
| RR | Number of breaths taken | Resting rate: 12-20 |
| | per minute | breaths per minute |
| | | (adults) |
| Step Counts | Number of steps taken, | Varies widely based on |
| | typically measured by a | individual activity level |
| | pedometer or accelerometer | |
| Body Temperature | The internal temperature of | Normal range: 36.5° C.- |
| | the body | 37.5° C. (97.7° F.-99.5° F.) |

Additionally, the EmbracePlus features real-time data collection capabilities, a 7-day battery life, and a waterproof design, making it highly suitable for continuous monitoring in everyday environments. The device integrates seamlessly with cloud-based platforms, ensuring that physiological data is captured, stored, and synchronized with minimal disruption to participants. This advanced functionality allowed for precise tracking of participants' emotional and physiological responses, offering valuable insights into the correlations between emotional states and their corresponding physical markers throughout the study.

By using the EmbracePlus in conjunction with daily and weekly self-reported emotional reflections, a comprehensive dataset for emotion prediction could be collected, with a focus on detecting anxiety as a key emotional state.

1) Baseline Data Collection: The following activities were conducted at the start of the residency week to establish baseline data.
i) Distribution of Devices: Participants were given EMPATICA EmbracePlus devices the night before the start of the residency week. They were instructed to wear the devices continuously throughout the residency week and throughout the semester to ensure continuous physiological data collection.
ii) Mindfulness Activities: During the in-person session, participants were guided through focused breathing techniques as a mindfulness exercise to induce a relaxed state. These activities ensured that the baseline data captured minimal stress levels, providing a clear contrast for future data analysis.
iii) Psychological Assessments: Participants completed the Perceived Stress Scale (PSS) and World Health Organization Well-Being Index (WHO-5). These assessments were administered twice during the residency week, once before arrival on campus and once after returning home. This allowed for the measurement of perceived stress levels and overall mental well-being at baseline.

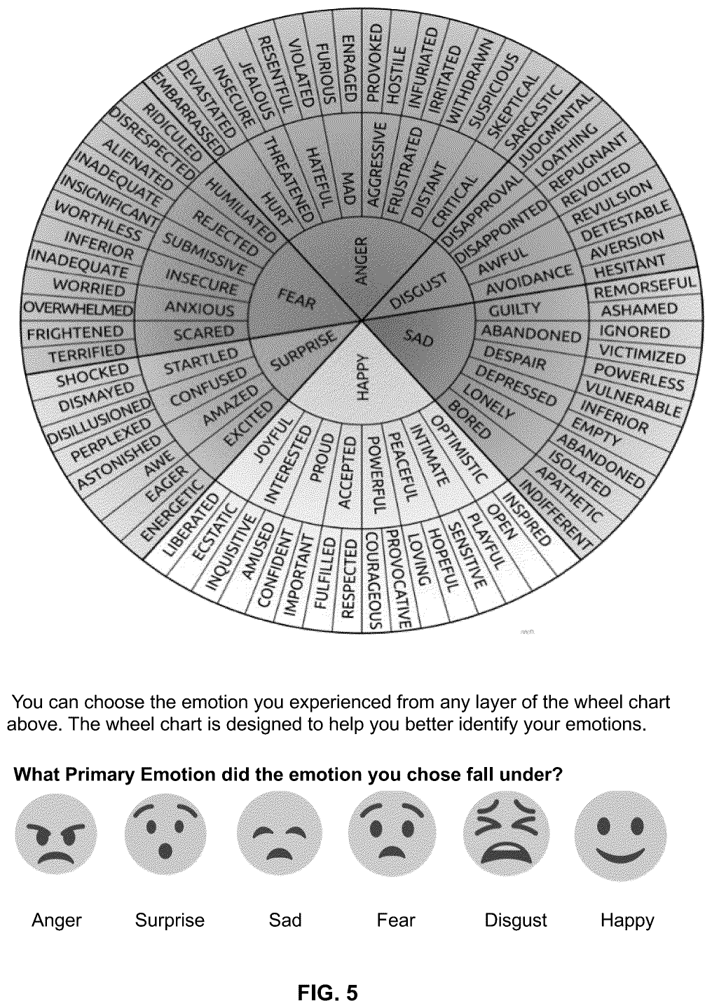

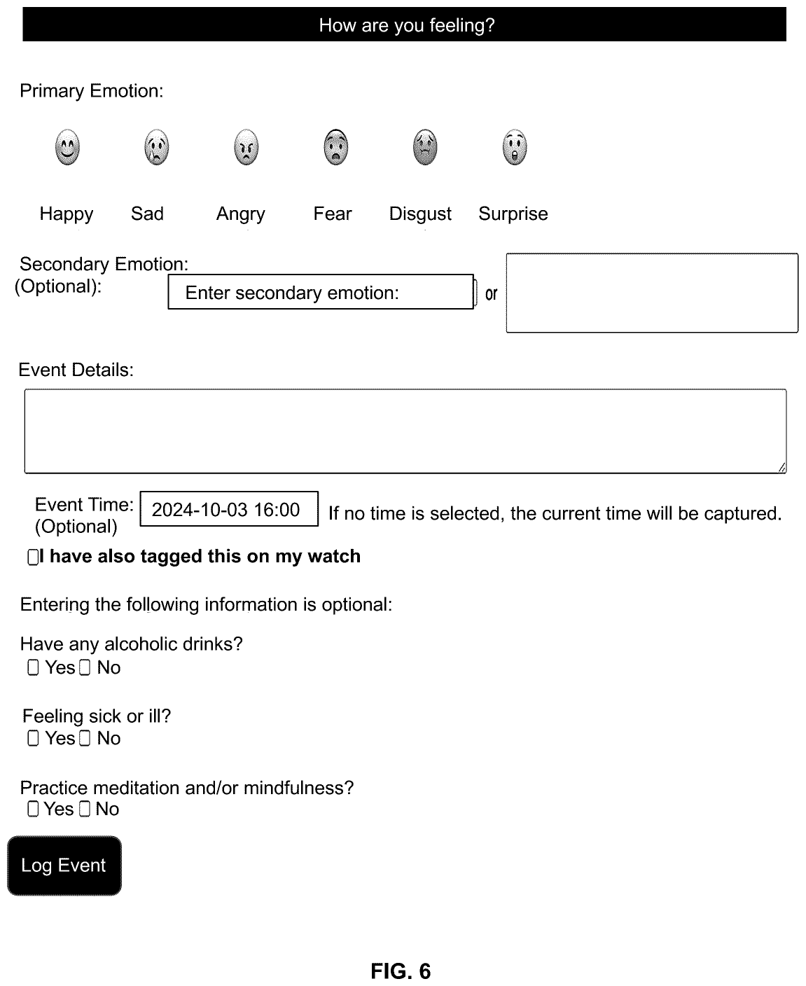

2) Residency Week: Intensive Data Collection: During the residency week, participants were required to report their emotions daily using structured reflection prompts. These daily reflections captured real-time emotional states, providing a detailed account of their emotional experiences throughout the day.
i) Daily Reflections: Participants used structured daily reflection prompts to document their emotional experiences. They were instructed to select emotions using an emotion wheel and describe the physical and emotional impacts of key events. FIG. 5 shows an example of reflection prompts.
ii) Web Application for Real-Time Logging: In addition to the daily reflections, participants had access to a custom web application that allowed them to log emotions in real time. This application enabled participants to report emotions beyond the structured prompts, recording their emotional states throughout the day (FIG. 6).
iii) Physiological Monitoring: Participants continuously wore the EMPATICA EmbracePlus device throughout the residency week, which recorded various physiological metrics, including PR, skin temperature, EDA, RR, and pulse rate variability (PRV).

3) Post-Residency: Ongoing Monitoring and Reflection: Following the residency week, participants shifted from daily reflections to weekly reflections, where they documented their emotional states and experiences once a week. Additionally, participants completed PSS and WHO-5 assessments on a bi-weekly basis. The Web application remained accessible throughout the semester, allowing participants to continue to log emotions in real-time as they occurred. This ongoing real-time log complemented weekly reflections and provided continuous emotional data for further analysis.
i) Weekly Reflections: In the post-residency period, participants completed weekly reflections using the same structured prompts, allowing continued documentation of emotional states and significant experiences throughout the semester.
ii) Web Application: The custom web application, which was used during the residency week, remained available throughout the semester. Participants used it to capture real-time emotions as they occurred, providing a continuous stream of emotional data to complement weekly reflections.

4) Data Synchronization and Logging: The physiological data collected by the EMPATICA EmbracePlus device was regularly synchronized to a secure cloud environment. This synchronization process was critical to ensure that objective physiological metrics could be directly correlated with subjective emotional experiences reported by participants. By aligning the timestamped physiological data with emotional events and reflections, the study was able to capture the realtime physiological manifestations of emotional states.

The synchronization process involved matching the precise timestamps of the physiological signals with the emotional reflections and tagged events recorded by participants. This real-time synchronization provided a comprehensive dataset, allowing for in-depth analysis of how specific emotional states, such as anxiety and fear, were reflected in physiological markers.

All data, including physiological signals, self-reported emotional reflections, and psychological assessment scores, were de-identified, securely logged, and stored for subsequent analysis. This robust dataset provides valuable insights into the correlations between physiological responses and emotional states, which will serve as the foundation for building predictive machine learning models.

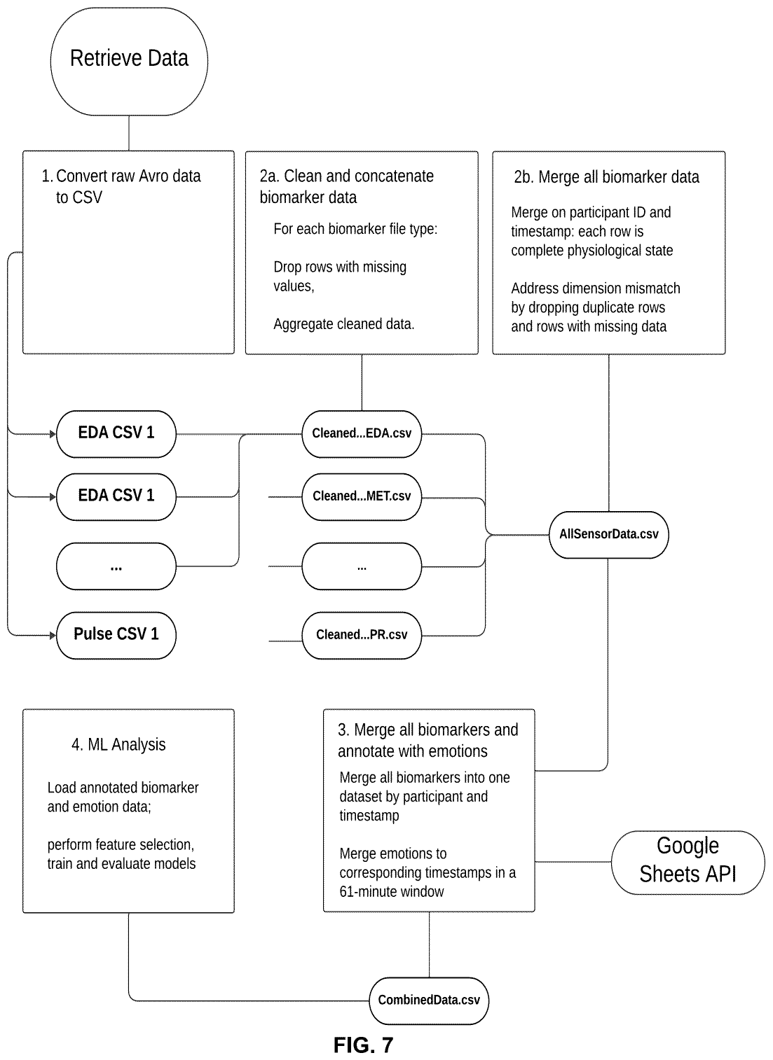

5) Data Pre-processing: To ensure that the data were ready for analysis, several pre-processing steps were performed on the physiological data downloaded from the EMPATICA cloud via Cyberduck. For this study, data collected during the residency week was examined, during which participants' emotions were closely monitored (FIG. 7).
i) Data Conversion: The raw physiological data was initially stored in the. avro format. Using a Python script, these files were converted into the more manageable. csv format, enabling easier manipulation and analysis. This conversion process was optimized for large datasets, utilizing parallel processing to ensure efficient handling of data from multiple participants.
ii) Noise Reduction and Data Cleansing: The data cleansing process involved identifying and removing rows containing missing values, particularly those flagged with a missing value reason column. Once these rows were removed, the column itself was dropped. This step ensured that only complete and valid data points were retained for analysis, thereby improving the overall quality of the dataset.
iii) Normalization: To ensure comparability across different participants, the physiological data was normalized. Standardizing the biomarker data allowed for consistent analysis, enabling the model to better detect deviations related to emotional states. The results were a set of cleaned digital biomarker files.
iv) Synchronization with Reported Emotional Data: Before integrating emotional data, the various physiological sensor datasets was merged. These individual files were then combined into a master dataset. This involved renaming columns for consistency, ensuring accurate matching of Participant_ID and Timestamp fields, and eliminating any unnecessary columns. A critical step in the preprocessing phase involved aligning the physiological data with the emotional data reported by participants. This synchronization was achieved by matching timestamps and participant IDs from the physiological signals with the emotional events logged via the custom web app and weekly assessments. By aligning data points at the minute level, the dataset was able to link real-time physiological responses with the self-reported emotional states, ensuring precise and accurate analysis.
v) Data Cleansing and Aggregation: After synchronization, additional data cleansing steps were performed to remove any remaining incomplete or inaccurate records.

A wide range of physiological signals and values were included in the final dataset:
EDA—‘eda scl usiemens’,
PR—‘pulse rate bpm’,
RR—‘respiratory rate brpm’,
Skin Temperature—‘temperature celsius’, and
Accelerometry (Standard Deviation)—‘accelerometers std g accelerometers std’,
Activity Counts (X-axis)—‘counts x axis’,
Activity Counts (Y-axis)—‘counts y axis’,
Activity Counts (Z-axis)—‘counts z axis’,
Vector Magnitude—‘vector magnitude’,
Activity Class (Categorical)—‘activity class’,
Step Counts—‘step counts’,
PRV, RMSSD—‘prv rmssd ms’,
Accelerometry (MET Standard Deviation)—‘accelerometers std g met’, and
MET—‘met’.

Additionally, the dataset also includes:
Primary Emotion—‘Primary Emotion’,
Secondary Emotions—‘Secondary Emotions’,
Event Details—‘Event Details’,
Watch Tag—‘Watch Tag’,
Alcohol Consumption—‘Alcohol’,
Sickness—‘Sickness’,
Mindfulness Activity—‘Mindfulness’, and
Fear Indicator—‘is fear’.

These preprocessing steps ensured that the data was clean, standardized, and accurately synchronized, laying the groundwork for developing machine learning models capable of predicting emotional states, particularly anxiety.

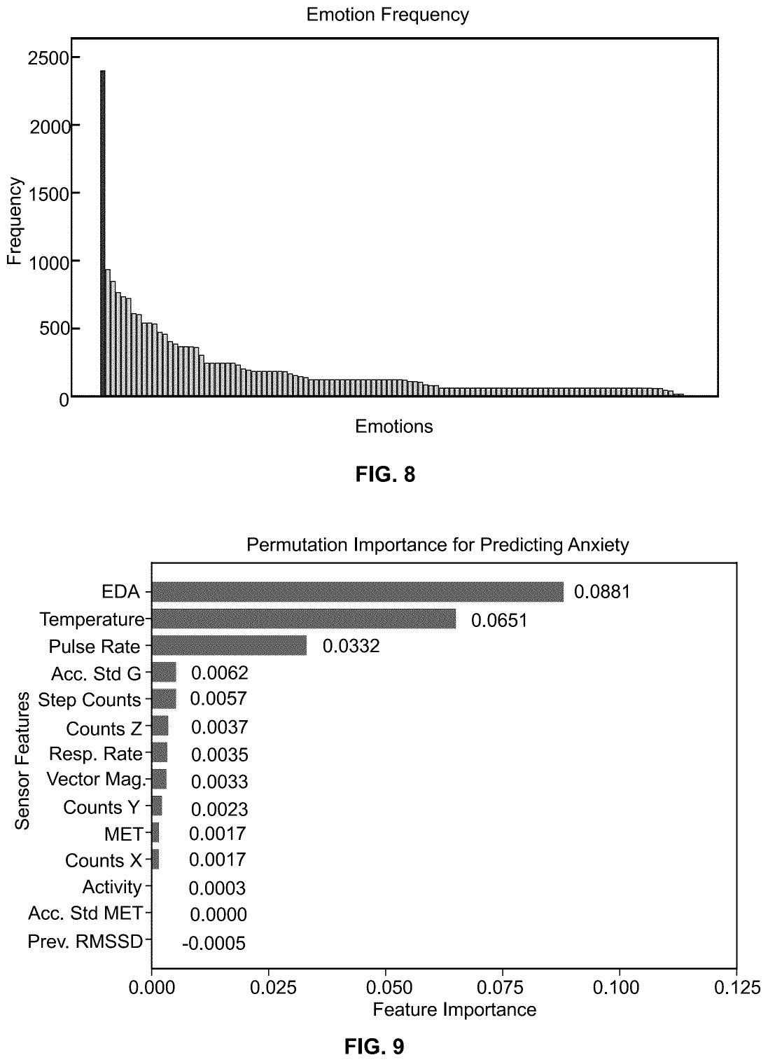

6) Feature Selection: The plot in FIG. 8 shows the relative frequencies of self-reported secondary emotions in the participant data, with anxiety marked in red. The primary goal of the study was to identify the most relevant physiological features for accurately predicting anxiety as an emotional state.

Throughout the study, participants consistently logged their emotional states. Consequently, the feature selection process was designed to enhance the model's ability to detect anxiety related physiological responses, with a particular focus on predicting anxiety.

Several feature selection techniques were examined to identify the most important features for the model. Permutation importance was chosen as the final method, recognizing its model-agnostic nature and robustness. The methods are outlined below:

1) Correlation Analysis: correlation analysis was initially conducted to assess the relationships between sensor features and the target variable, is anxiety. While useful for understanding basic relationships, it was only exploratory.

2) Variance Thresholding: A variance threshold was applied to remove features showing little variation across participants or during anxiety-related events. While it helped simplify the dataset, it was not the final method used for feature selection.

3) Recursive Feature Elimination (RFE): RFE was used to train machine learning models and iteratively eliminate the least important features. Although it highlighted key features, RFE was discarded due to its dependence on specific model structures.

4) Correlation-Based Feature Selection (CFS): This method was tested to remove highly inter-correlated features to avoid redundancy. While it was useful, it didn't offer the same flexibility and reliability as permutation importance.

5) Permutation Importance: Ultimately, we selected permutation importance as the final method. By randomly shuffling feature values and measuring the decrease in model performance, this method provided a robust, model-independent assessment of feature importance. It performed consistently well across models such as Random Forest and XGBoost, making it the most reliable choice.

Final Selected Features: Through the feature selection process, the following key physiological signals were identified as the most predictive for detecting anxiety-related emotional states:
i) EDA (eda scl usiemens): Measured in microsiemens, this signal captures the skin's conductance, reflecting the autonomic nervous system's response to emotional stimuli.
ii) PR (pulse rate bpm): Measured in beats per minute, PR provides insights into cardiovascular responses linked to heightened emotional states.
iii) Skin Temperature (temperature celsius): Measured in degrees Celsius, skin temperature is a critical marker of emotional arousal, with fluctuations indicating stress or relaxation.

These features were selected for their strong ability to capture physiological responses associated with anxiety and were subsequently used to develop machine learning models for anxiety prediction.

As depicted in FIG. 9, the feature importance graph highlights the weight each feature contributed to the model's performance. Permutation importance was used to evaluate and confirm the relevance of these features in the anxiety detection model.

7) Machine Learning Techniques: Various machine learning models and ensemble techniques were applied to predict the emotional state of anxiety, with each model's performance evaluated using metrics such as accuracy, precision, recall, and F1 score. The models included both traditional classifiers and ensemble approaches that utilized advanced techniques for addressing class imbalance and optimizing performance. These methods incorporated the Synthetic Minority Over-sampling Technique (SMOTE), Anomaly Detection-based Sampling for the Newcomers (ADYSN), threshold tuning, cost-sensitive learning, focal loss, and data cleaning techniques like Tomek Links combined with Edited Nearest Neighbors (ENN).

The models and configurations evaluated included:
i) RandomForest and BalancedRandomForest with Cost-Sensitive Learning;
ii) ADYSM (RandomForest and EnsembleModel);
iii) EnsembleModel with class weights, focal loss, and threshold tuning;
iv) Hybrid Ensemble models using threshold tuning and Tomek Links+ENN for class balancing;
v) Stacking Ensemble with optimized threshold tuning;
vi) XGBoost Model and BaggingClassifier with Cost-Sensitive Learning; and
vii) Tuned RandomForest with hyperparameter tuning and optimized thresholds.

The performance outcomes, detailed in Table 3, highlight how each model and balancing strategy contributed to the effectiveness of anxiety prediction.

TABLE 3
Performance comparison of machine learning models

| Model | Method | Accuracy | Precision | F1 Score |
|---|---|---|---|---|
| Random Forest | ADYSN | 82.00% | 0.51 | 0.54 |
| EnsembleModel | ADYSN | 82.07% | 0.51 | 0.55 |
| EnsembleModel | Class Weights | 85.42% | 0.68 | 0.50 |
| EnsembleModel | focal loss | 85.54% | 0.70 | 0.49 |
| EnsembleModel | Threshold Tuning | 83.39% | 0.55 | 0.54 |
| Hybrid Ensemble | Threshold Tuning | 82.94% | 0.54 | 0.54 |
| Hybrid Ensemble | Tomek Links + ENN | 73.63% | 0.39 | 0.51 |
| Stacking Ensemble | Threshold Tuning | 77.18% | 0.43 | 0.53 |
| XGBoost Model | Cost-Sensitive | 73.94% | 0.39 | 0.51 |
| | Learning | | | |
| Balance RandomForest | Cost-Sensitive | 75.63% | 0.41 | 0.53 |
| | Learning | | | |
| BaggingClassifier | Cost-Sensitive | 83.05% | 0.68 | 0.24 |
| | Learning | | | |
| XGBoost | Cost-Sensitive | 85.16% | 0.68 | 0.47 |
| | Learning | | | |
| TuneRandomForest | Hyperparameter | 85.76% | 0.74 | 0.47 |
| | Tuning | | | |
| TuneRandomForest | Optimized Threshold | 81.36% | 0.49 | 0.56 |
| Random Forest | Cost-Sensitive | 84.07% | 0.58 | 0.52 |
| | Learning | | | |

Key findings from model evaluations include:
i) RandomForest (ADYSN, Selected Features): Achieved an accuracy of 82.00% and an ROC AUC of 81.56%. This configuration yielded strong precision (0.9) and recall (0.88) for non-anxiety cases (Class 0) but had moderate sensitivity for anxiety (Class 1) with an F1 score of 0.54.
ii) EnsembleModel (Class Weights, Selected Features): Performed well with an accuracy of 85.42% and an ROC AUC of 83.60%. While it achieved a high recall (0.96) for non-anxiety cases, it struggled with Class 1, with lower recall (0.39) but higher precision (0.68), suggesting a slight bias toward non-anxiety predictions.
iii) EnsembleModel (focal loss, Selected Features): Similar to the class-weighted model, this configuration obtained an accuracy of 85.54% and balanced high recall for Class 0 (0.96) with moderate precision for Class 1 (0.7), though recall for Class 1 remained lower (0.37).
iv) Hybrid Ensemble (Tomek Links+ENN, Selected Features): This model, which incorporated Tomek Links and Edited Nearest Neighbors for balancing, achieved an accuracy of 73.63% with an ROC AUC of 82.18%. Precision for Class 0 was high (0.93), but sensitivity for Class 1 was limited, with a recall of 0.74.
v) Stacking Ensemble (Threshold Tuning, Selected Features): This configuration reached an accuracy of 77.18% and an ROC AUC of 82.59%. Precision for Class 0 remained high (0.92), though sensitivity for anxiety detection (Class 1) saw moderate gains with a recall of 0.69.
vi) Tuned RandomForest (Optimized Threshold, Selected Features): Yielded an accuracy of 81.36% with an ROC AUC of 83.24%, maintaining high recall for Class 0 (0.85) and improved recall for Class 1 (0.64) with an F1 score of 0.56, suggesting effectiveness for balanced prediction between anxiety and non-anxiety cases.
vii XGBoost Model (Cost-Sensitive Learning, Selected Features): Demonstrated balanced performance with an accuracy of 73.94% and an ROC AUC of 82.82%. Recall for Class 1 was 0.74, indicating effective anxiety detection, though with a trade-off in Class 0 sensitivity.
viii) BalancedRandomForest (Cost-Sensitive Learning, Selected Features): With an accuracy of 75.63% and an ROC AUC of 83.08%, this model provided stable performance, balancing precision and recall across classes, and improving sensitivity to anxiety cases with a recall of 0.74.
ix) Each model configuration and balancing strategy influenced anxiety detection differently. The results, summarized in Table 3, provide insight into the trade-offs in precision and recall across both classes, with some models achieving high sensitivity to anxiety, while others maintained a bias towards non-anxiety predictions.

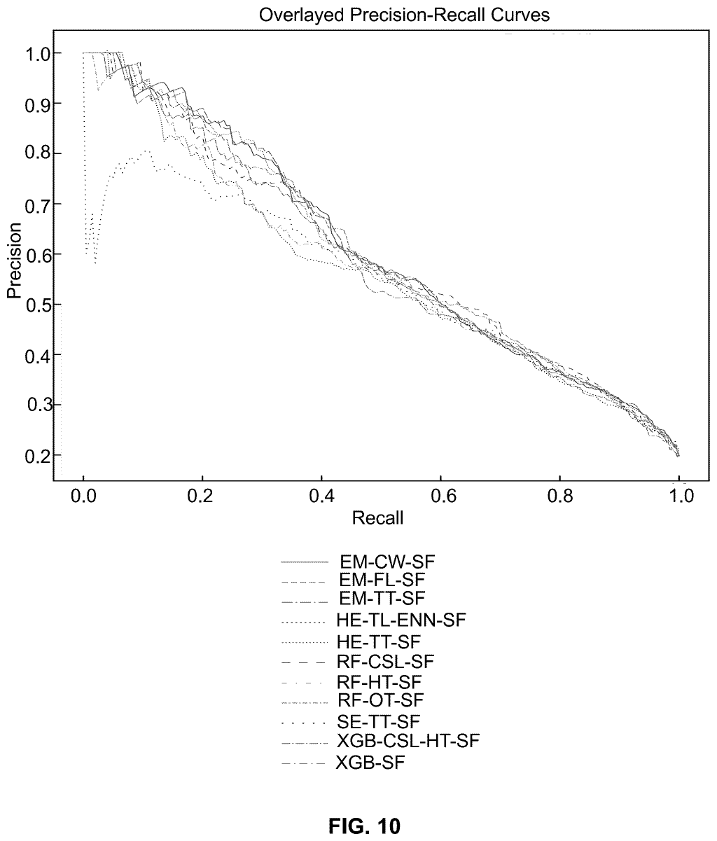

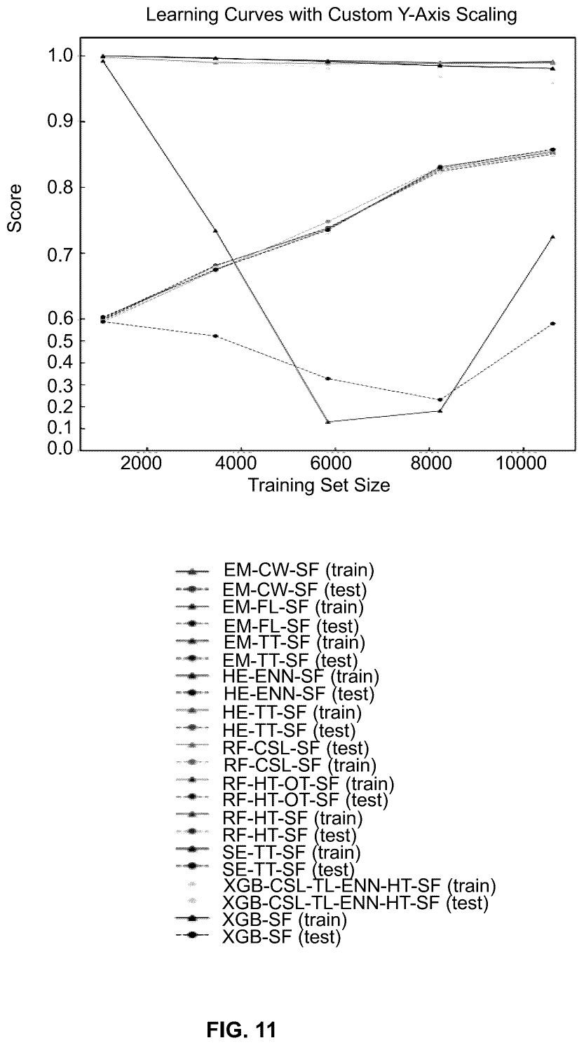

The abbreviations used in the model performance comparison figures (FIGS. 10-11) help clarify the models and techniques applied in the study. Table 4 and Table 5 outline what each abbreviation stands for.

TABLE 4
Abbreviations for training and data balancing techniques

| Abbreviation | Description |
|---|---|
| SF | Selected Features - The model was trained using the refined |
| | set of features that were chosen through feature selection |
| | methods |
| AF | All Features - The model was trained using the full dataset |
| | without feature reduction |
| SM | SMOTE Applied - SMOTE was used to address class |
| | imbalance |
| ADYSN | ADYSN Applied |
| CW | Class Weights - The model utilized class weighting to |
| | handle imbalance |
| FL | Focal Loss - Applied to focus the model's attention on |
| | hard-to-classify instances |
| TT | Threshold Tuning - Used to optimize the decision threshold |
| TL-ENN | Tomek Links and Edited Nearest Neighbors - A |
| | combination of techniques for cleaning and balancing data |
| CSL | Cost-Sensitive Learning - A technique that adjusts model |
| | loss based on class costs. |
| HT | Hyper parameter Tuning - Adjusting model parameters for |
| | optimal performance |
| OT | Optimized Threshold - Adjusting thresholds specifically for |
| | the RandomForest model |

TABLE 5
Abbreviations for machine learning models

| Abbreviation | Description |
|---|---|
| RF | Random Forest |
| ERF | BalancedRandomForest (Enhanced Random Forest) |
| BC | BaggingClassifier |
| EM | EnsembleModel |
| HE | Hybrid Ensemble |
| SE | Stacking Ensemble |
| XGB | Extreme Gradient Boosting (XGBoost) |
| LGBM | Light Gradient Boosting Machine (LightGBM) |
| HistGB | Histogram-Based Gradient Boosting |
| ERT | Extremely Randomized Trees (ExtraTrees) |

These abbreviations are used to show how different feature selection methods and class balancing techniques were applied to each model during the prediction process.

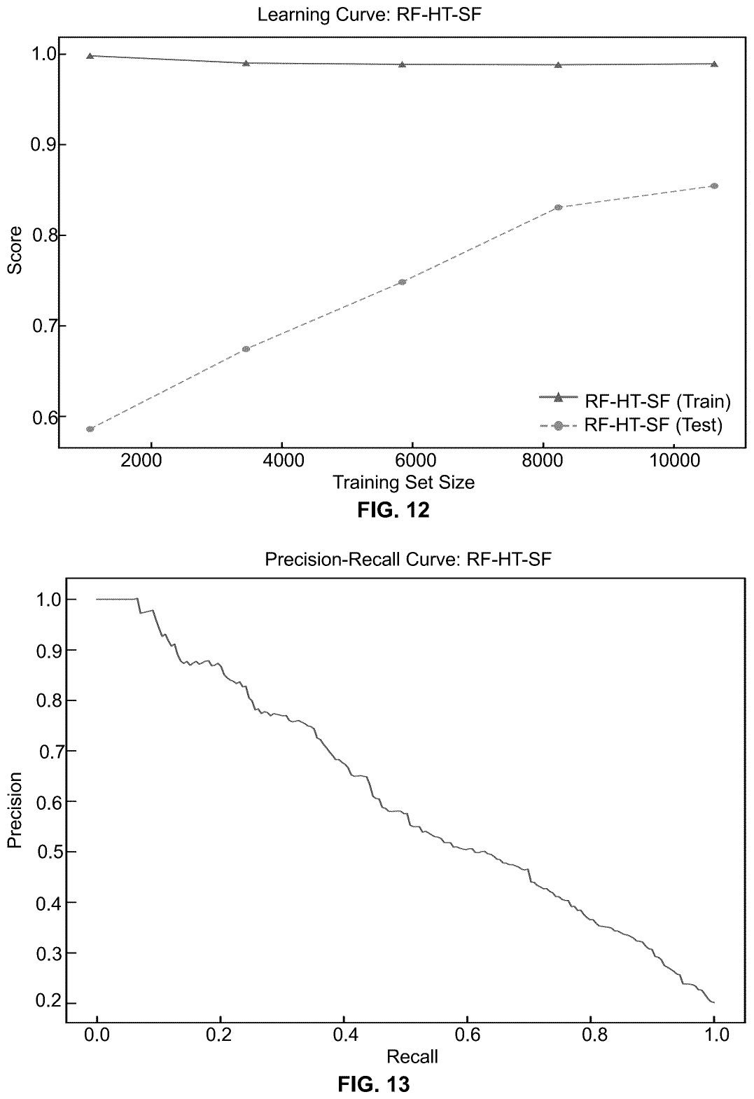

8) Model Insights: The Tuned Random Forest model with hyperparameter optimization emerged as the most balanced and effective configuration for predicting anxiety, delivering robust performance across both anxiety and non-anxiety classes. While other models, such as XGBoost and Ensemble techniques, showed promise-particularly when combined with cost-sensitive learning and threshold tuning-the Tuned Random Forest model consistently achieved the highest accuracy and balanced precision and recall (FIGS. 12-13).

Notably, models like the Hybrid Ensemble with Tomek Links+ENN and the Ensemble Model with focal loss also performed well, though they presented trade-offs between precision and recall in detecting anxiety. The BalancedRandomForest with cost-sensitive learning provided stable sensitivity for anxiety, indicating the effectiveness of cost-sensitive methods for this dataset.

Overall, models that utilized class balancing techniques such as ADYSN, cost-sensitive learning, and optimized thresholds demonstrated the most stable and accurate results. These findings highlight the significance of targeted sampling and tuning methods in enhancing predictive performance on imbalanced datasets, especially for nuanced emotional states like anxiety.

### Results

This study highlights the effectiveness of machine learning models in predicting emotional states, with a specific focus on anxiety. An analysis demonstrated that the Tuned Random Forest model with Hyperparameter Tuning achieved the best balance between accuracy, precision, and recall. This model configuration provided strong performance, with an accuracy of 85.76% and an AUC score of 83.24%, underscoring its robustness in detecting anxiety within an imbalanced dataset.

Key performance metrics for the Tuned Random Forest model were as follows:
1) Accuracy: 85.76%,
2) ROC AUC: 83.24%,
3) Precision (anxiety): 0.74,
4) Recall (anxiety): 0.35, and
5) F1-Score (anxiety): 0.47,

These results underscore the model's ability to predict anxiety with high accuracy and a balanced trade-off between precision and recall. While the recall for anxiety (Class 1) is moderate, this model's overall performance indicates that hyperparameter tuning was effective in optimizing sensitivity and precision.

### Example 3

Integration of Emotional Intelligence-Based Interventions Into PTSD Treatments Participants and contingencies

Military personnel are the primary subjects, ensuring the applicability to the targeted demographic. 50 participants are recruited.

Participant Recruitment, Retention and Attrition: In the event the number of participants is less than 50, contingency plans include offering incentives to participants to enhance recruitment rates and ensure an adequate sample size. Should participant dropout during the intervention period occur, a comprehensive engagement strategy, including regular communication, reminders, and support to maintain participant involvement, is implemented. Should participant attrition over time occur during longitudinal studies, a system is established for maintaining contact with the participants including regular check-ins and incentives for continued participation.

Emotion Labeling Challenges: Identifying the six primary emotions accurately may be challenging for participants, emotion labeling is simplified into three categories, including baseline, positive irregularity, and negative irregularity, and developing classes or instructions for participants. If this approach proves ineffective, further simplification into two categories, baseline, and irregularity, are considered to ensure the reliability of emotional data analysis.

Machine Learning Model Complexity: If the machine learning model's complexity becomes a barrier to interpretability, alternative, more interpretable algorithms such as decision trees or linear regression and visualization techniques such as feature importance plots and partial dependence plots enhance the model's transparency.

Data Quality and Variability and Security: Variability in self-reporting and data quality could pose challenges. To mitigate this, clear instructions are provided and participants are engaged in regular communication to ensure accurate data collection and minimize inconsistencies. Additionally, rigorous data quality control measures are employed during analysis to account for potential variations. IRB implemented stringent data security measures are followed with contingency plans in place for handling data breaches or security concerns. Data quality checks and engagement strategies are implemented to maintain the integrity of the longitudinal data.

Intervention Effectiveness: Some participants may struggle to adhere to the assigned mindfulness interventions. To mitigate this, regular check-ins and reminders are implemented to enhance participant engagement and adherence to the interventions. Additionally, alternative mindfulness activities are offered to accommodate individual preferences and needs. There may be variations in the effectiveness of mindfulness interventions. To address this, the progress is continuously monitored and interventions are adapted as necessary to optimize their impact. Additionally, ongoing communication with participants is maintained to gather feedback and make real-time adjustments to the intervention programs.

Feedback Collection: Obtaining consistent and meaningful feedback from participants may be challenging. In case of inadequate feedback, the feedback collection methods and conducting additional participant engagement activities may be adapted to encourage valuable input.

Model Optimization: If the reinforcement learning model does not perform as expected, iterative model optimization is implemented and alternative machine learning techniques are explored to achieve the desired results.

### Sample Size and Data Collection Methods

During baseline assessment, data is collected from a diverse sample of 50 participants for at least 12 months, for example, 12 to 18 months, including military service members, veterans, and civilians. This sample size is chosen to ensure there is a high likelihood of identifying significant differences in the outcomes if they exist in the population being investigated. Moreover, these arrangements result in the collection of 1200 data points in the VR-inducing emotion cases and 5200 data points in the participants tagging two emotion-deregulating events per week in their day-to-day lives. Using Wilson Score intervals, the former has a sensitivity and specificity 95% confidence interval of ±2.5%, and the latter has 95% confidence interval of ±1.5%. The consecutive months results in 1200, and 1300 data points for baseline and PTSD groups. Using Wilson Score intervals, the 95% confidence intervals are ±2.5% around the calculated sensitivity and specificity values of Machine Learning II.

Data is collected through self-report questionnaires, for example, the Big Five Personality Test, the Eysenck Personality Questionnaire (EPQ), The Trait Emotional Intelligence Questionnaire (TEIQue), the Affect Intensity Measure (AIM), Clinician-Administered PTSD Scale (CAPS) for PTSD symptoms, EQ-i 2.0, and ESCI 360 for emotional intelligence), wearable device data (e.g., heart rate variability, sleep patterns), and qualitative interviews to gather participant feedback.

Statistical analysis is conducted using t-tests, chi-squared tests, and mixed-effects models, to compare outcomes between the intervention and control groups. Longitudinal data is analyzed using repeated measures ANOVA to assess changes over time. Qualitative data from interviews is analyzed thematically. The following evaluation criteria and metrics are used:
() Reduction in PTSD symptoms (measured by the PCL-5),
ii) Improvement in emotional intelligence (measured by the EQ-i 2.0 and ESCI 360),
ii) Engagement with and adherence to the personalized EI interventions,
iv) Participant feedback on the acceptability and perceived effectiveness of the interventions, and
Long-term maintenance of reduced PTSD symptoms in the follow-up phase.

### Correlation of Physiological Markers With Emotional States

Participants undergo an initial assessment of PTSD symptoms, EQ-i 2.0, ESCI 360 assessments, and baseline assessments, such as WHO-5, PSS, STAI, HAMD, CAPS/PCL-5, open-ended journal prompts, and demographic characteristics. In recent years, an increased demand for improving mental health has prompted the use of emerging technologies, most notably, Virtual Reality (VR). Leveraging the immersive capabilities of VR, participants are exposed to controlled and safe virtual environments designed to induce specific emotional responses. The VR environments are customized based on the principles of emotion induction to establish a benchmark, mapping EI and physiological signals in a VR setting, while exploring the relationship between EI and reduction of PTSD symptoms.

Sensitivity and specificity targets are established, ensuring that the model achieves a minimum sensitivity level, e.g., 80% for correctly identifying emotions, and a minimum specificity level, e.g., 85% for correctly identifying primary emotions. These thresholds serve as critical benchmarks for validating the accuracy and effectiveness of the model in identifying who may benefit from PTSD interventions while minimizing false alarms.

1) Data Collection: Participants are equipped with wearable devices, such as EMPATICA EmbracePlus, Fitbit Sense 2, and VR headsets for an immersive experience. The wearables continuously monitor physiological data, including vital indicators like heart rate, blood oxygen saturation (SpO2), sleep patterns, electrodermal activity (EDA), skin conductance level (SCL), and temperature. The watches operate throughout both the daytime and nighttime, collecting data at high-frequency intervals to provide high-resolution physiological data corresponding to one's emotions. Participants are expected to wear the devices for a minimum of 8 hours daily, ensuring a holistic capture of their physiological responses. Alongside, the VR headset offers a range of virtual experiences, tailored to invoke emotions both in lab settings and in daily scenarios.

2) Emotion Labeling: Within the VR environment, participants experience the six primary emotions (happiness, surprise, anger, sadness, disgust, and fear) in different scenarios, through which they engage in a self-reporting process to identify their emotions using a predefined set of primary emotions and after specific emotion inducing emotions are shown within the VR setting. This setup offers the advantage of providing immediate feedback after the VR experience, ensuring more accurate emotional labeling.

Additionally, participants engage in a weekly self-reporting process to identify their emotions using a predefined set of primary emotions (such as happiness, surprise, anger, sadness, disgust, and fear). Participants in the cohort also report emotion deregulation events that can be labeled by “Freez” and “Appease”, terms widely used in the PTSD context, and create a total of eight different labels. This weekly reflection allows participants to recap their experiences over the past week and recognize their emotional fluctuations. Participants tag specific events when their emotional states deviate from their baseline, offering a deeper understanding of emotions within the context of real-life events and variations throughout the week. Fifty participants undergo the VR emotion induction sessions twice where each primary emotion is induced twice collecting 1200 data points. Fifty participants wear the wearables for a year tagging two emotion deregulating events and collecting 5200 data points.

3) Machine Learning Model I Implementation: Utilizing the physiological data as the independent variable and self-reported emotional states as dependent variables, the machine learning model is implemented to find the relationships and connections between vital signs and emotional states. Convolutional Neural Networks (CNNs), Long Short-Term Memory (LSTM), and Recurrent Neural Networks (RNNs) are the potential algorithms due to their effectiveness in sequence and temporal pattern recognition. Additionally, Random Forests and Support Vector Machines (SVMs) are considered for their proficiency in identifying nonlinear relationships within multidimensional datasets, and more post-modeling techniques, such as feature importance analysis, SHAP, LIME values, and visualization (e.g. partial dependence plots) are employed to enhance the interpretability of the complex predictions, which makes the hidden patterns more accessible for human understanding and validation.

### Evaluation of Mindfulness and Emotional Intelligence (EI) Interventions

There are two cohorts for comparison: a baseline group representing a general population sample and a PTSD cohort with varying levels of symptom severity. This design allows for a nuanced analysis of the interventions' efficacy across different emotional and psychological starting points. Participants in each of the above two groups are randomly assigned to either the personalized EI-based intervention group or a control group, creating four distinct groups that ensure a balanced representation of participants' ability to regulate their emotions as the result of EI-based intervention. All participants initially complete the EQ-i 2.0 and ESCI 360 assessment to gauge baseline EI. The participants in the control group receive standard care without any specific EI-focused interventions. For this study, standard care within the control group consists of usual support without the addition of specific EI-focused methods. This may include general wellness advice and access to standard mental health resources, representing the baseline against which the intervention's efficacy is assessed.
i) Personalized EI Interventions: Those in the intervention group use wearable devices and machine learning algorithms to partake in EI training tailored to their needs. They also collaborate with an EI coach to enhance specific EI competencies, choosing from a suite of mindfulness activities.
ii) Control Group: Participants in this group continue with standard care, which lacks the tailored EI focus present in the intervention group.

To study the trends in participants' emotional state and habitual emotional patterns vs traits, instantaneous and long-term observations are carried out. Each one of the four cohorts are constituted of 25 participants.

The baseline groups undergo stressful conditions such as observing stressful imagery via VR, encouraging them to recall a past stressful event, and having them complete a task under time pressure. Participants' ability to regulate their emotional state are compared between the EI-based intervention group and the control group. The emotional states are measured using wearable watches and the algorithms to determine emotional irregularities. Each participant is invited to four sessions, during 12 months, undergoing twelve emotion-deregulating sessions. As such 1200 data points are generated per cohort. The PTSD group does not undergo this study. The above study assesses the effectiveness of mindfulness and emotional intelligence-based interventions to regulate one's emotions.

The PTSD groups are monitored long-term, for 12 months, to assess their emotional traits. Both the EI-based intervention and the control group undergoes regular and routine tests such as The Trait Emotional Intelligence Questionnaire (TEIQue), which assesses emotional self-efficacy, the Affect Intensity Measure (AIM), which gauges the strength or weakness of a person's emotional responses, and Clinician-Administered PTSD Scale (CAPS): Considered the “gold standard” in PTSD assessment, the CAPS is a structured interview that assesses the frequency and intensity of PTSD symptoms. The PTSD groups use the wearable watches to continuously measure their physiological signals and emotional state. PTSD cohorts use open-ended prompts to capture any emotional events and the EI or non-EI-based interventions that they take part in.

1) Intentional Practice: The EI-based intervention groups in the PTSD and baseline groups are introduced to different mindfulness techniques, such as meditation, EI-based strategies, and breathing exercises, through guided sessions. The PTSD groups track their EI-based interventions and events with open-ended prompts. Each participant records one emotion-deregulating event per week and the interventions they used to regulate their emotions. Doing so captures 1300 data points per cohort over a year.

2) Mindfulness Sessions: PTSD groups engage in regular therapy sessions. The first PTSD group focuses on mindfulness interventions and the other on general aspects of well-being over 12 months. Subsequently, in collaboration with a designated coach, participants focus on enhancing a specific EI competency. This collaborative effort results in a customized list of 3 to 5 activities designed to bolster the targeted EI competency.

3) Machine Learning Model II Implementation: In the assessment of intervention effectiveness, the model takes both subjective and objective measurements of interventional activities and emotional states as input. The outputs are the emotional state regulations in the baseline group and the emotional trait trends in the PTSD groups. The machine learning output includes patterns and correlations identified by the model. Machine learning algorithms such as Random Forest, Support Vector Machines (SVMs), and Neural Networks, are among the potential choices.

### References

1. Espinosa, et al. Psychological Trauma: Theory, Research, Practice, and Policy, 11(1):28, 2019.
2. Arora et al., Sleep Medicine Reviews, 62:101593, 2022.
3. J. Block, The challenge of response sets: Unconfounding meaning, acquiescence, and social desirability in the MMPI, 1965.
4. L. K. McCorry, American Journal of Pharmaceutical Education, 71(4), 2007.
5. Campanella, et al., Sensors (Basel), 23(7):3565 March 29 2023.
6. K. T. Johnson and R. W. Picard, Neuron, 108(1):8-12, October 14 2020.
7. Copurkaya, et al., Data Brief, 49:109297, August 2023.
8. Halimeh, et al., Seizure, 110:99-108, August 2023.
9. Picard et al., Neurology, 89(6):633-635, August 8 2017.
10. Comai, et al., Stud Health Technol Inform, 306:25-30, August 23 2023.
11. Zhang, et al., Int J Health Geogr, 22(1):15, June 21 2023.
12. Liu, et al., Improving mental health support in engineering education using machine learning and eye-tracking,” IEEE Frontiers in Education Conference (FIE), pp. 1-5, 2023.
13. Pipatpratarnporn, et al., Wrist-worn smartwatch and predictive models for seizures,” Epilepsia, July 28 2023.
14. Kaplan, et al., Journal of Business and Psychology, 29:367-380, 2014.
15. A. Di Fabio and M. E. Kenny, Frontiers in Psychology, p. 1182, 2016.
16. Iqbal et al., Sensors, 22(21):8135 October 2022.
17. Balconi, et al., Stress, 12:200-209, November 2018.
18. N. Ahmadi and T. Hammond, Recognizing and Responding to Human Emotions: A Survey of Artificial Emotional Intelligence for Cooperative Social Human-Machine Interactions, in 2023 IEEE Frontiers in Education Conference (FIE), pp. 1-5, October 2023.
19. Egger et al., Electronic Notes in Theoretical Computer Science, 343:35-55, May 2019.
20. A. Dessai and H. Virani, Emotion Detection Using Physiological Signals in 2021 International Conference on Electrical, Computer and Energy Technologies (ICECET), December 2021, pp. 1-4, December 2021.
21. A. Tripathi and T. Choudhury, IEEE International Conference on Contemporary Computing and Communications (InC4), 1:1-5, April 2023.
22. Kattenborn, et al. ISPRS Journal of Photogrammetry and Remote Sensing, 173:24-49, 2021.
23. A. Graves and A. Graves, “Long short-term memory,” Supervised sequence labelling with recurrent neural networks, pp. 37-45, 2012.
24. L. Breiman, Machine learning, 45:5-32, 2001.
25. W. S. Noble, Nature biotechnology, 24(12):1565-1567, 2006.
26. R. Bar-On, International Journal of Sociology and Social Policy, 29:164-175, 1997.
27. K. Dhakal, Journal of the Medical Library Association: JMLA, 110(2):270, 2022.
28. R. S. Sutton and A. G. Barto, Reinforcement learning: An introduction. MIT press, 2018.

What is claimed is:

1. A method for understanding and optimizing emotional states in a subject using a wearable device, comprising:
a) collecting physiological data from the subject using the wearable device;
b) receiving self-reported emotional states from the subject via a user interface in the wearable device;
c) processing the physiological data using a machine learning model to identify patterns or relationships or a combination thereof between said physiological data and said self-reported emotional states; wherein as the machine learning algorithm develops, reliance on self-reported emotions decreases as processing the physiological data increases; and
d) employing a reinforcement learning model; wherein the states in reinforcement learning algorithms are the emotions calculated using the developed machine learning algorithm.

2. The method of claim 1, wherein the wearable device comprises a watch, a ring, a patch or other wearable technology.

3. The method of claim 1, wherein said machine learning model comprises:
Convolutional Neural Networks (CNNs) configured for deciphering complex, time-dependent associations present in physiological signals;
Long Short-Term Memory (LSTM) and Recurrent Neural Networks (RNNs) configured for sequence and temporal pattern recognition; and
Random Forests and Support Vector Machines (SVMs) for identifying nonlinear relationships within multidimensional datasets.

4. The method of claim 1, wherein the wearable device comprises at least a PPG sensor, an ECG sensor, a bioimpedance sensor, a gyroscope, an accelerometer, a temperature sensor, and a force sensor to measure physiological signals comprising pulse rate, pulse rate variability, SpO₂, electrodermal activity, skin temperature, or actigraphy data or a combination thereof.

5. The method of claim 1, wherein the self-reported emotional states are primary emotions comprising happiness, fear, sadness, disgust, or anger or a combination thereof.

6. The method of claim 1, wherein in step d) the reinforcement learning model comprises activities defined as EQ enhancing activities, consuming nootropics, adopting new dietary habits, physical exercises, or other activity effective to improve mental status, or a combination thereof.

7. The method of claim 6, wherein in step d) the reinforcement learning model comprises rewards defined as trends in mental well-being metrics that are perceived stress test, who-5, concentration trends of mental status-related biomarkers in the blood, or physical attributes of the subject or other metric for assessing trends of mental or physical status over time.

8. The method of claim 7, wherein said reinforcement learning model scores the activities based on the rewards observed.

9. The method of claim 1, further comprising integrating a Return Decomposition for Delayed Rewards (RUDDER) framework into said reinforcement learning model to address issues of delayed rewards.

10. The method of claim 9, wherein RUDDER utilizes return decomposition techniques to assign credit to intermediate actions within a delay horizon.

11. The method of claim 1, further comprising improving emotional intelligence in the subject by optimizing at least one intervention that is implemented within the reinforcement learning model.

12. The method of claim 11, wherein the at least one intervention is an activity comprising mindfulness, mental imagery, breathing, body scanning, exercise or dietary changes.

13. A system comprising at least one wearable device configured to:
continuously measure physiological signals of a subject wearing the wearable device, said wearable device comprising a machine learning-based framework;
assess emotional states of the subject based on said physiological signals via the machine learning-based framework;
detect deviations in emotional states; and
utilize the results of at least one reinforcement learning model to suggest, in real time, activities to alter the subject's emotion, to enhance an emotional quotient (EI) or to reduce stress and improve overall well-being over time or a combination thereof.

14. The system of claim 11, further configured to utilize the results of at least one reinforcement learning model to suggest, in real time, food intake to alter the subject's emotion, to induce happiness or to reduce stress over time or a combination thereof.

15. The system of claim 11, further configured to utilize the results of at least one reinforcement learning model to suggest, in real time, physical or athletic activities to alter the subject's physical outcome over time comprising muscle mass or body mass index (BMI) or a combination thereof.

16. A method for predicting anxiety in a subject, comprising:
measuring continuously physiological signals in the subject wearing a wearable device comprising a plurality of sensors, a machine learning-based framework and a reinforcement learning model;
cleaning and concatenating physiological data derived from the physiological signals from the wearable device to form a complete physiological state;
assessing emotional states based on the measured physiological signals by merging the physiological data with corresponding emotional annotations over a predefined window of time via a method of data synchronization;
detecting a deviation in a stress-inducible emotional state in the subject indicative of an onset of anxiety; and
performing a machine learning analysis by selecting features from annotated physiological data and training a model to evaluate the emotional state, thereby predicting the onset of anxiety therefrom.

17. The method of claim 16, further comprising suggesting a personalized emotional intelligence intervention based on results from the reinforcement learning model to reduce or prevent anxiety in the subject.

18. The method of claim 17, wherein the emotional intelligence intervention is mindfulness.

19. The method of claim 16, wherein the cleaning and concatenating step comprises handling missing data, aggregating cleaned physiological data and merging the physiological data with a participant ID and timestamp.

20. The method of claim 16, wherein the subject suffers from post traumatic stress syndrome.

21. The method of claim 16, wherein the subject is in the military, is a veteran, is a civilian or is in an educational setting or a professional setting or a combination thereof.
